(19) United States
(12) Patent Application Publication
(10) Pub. No.: US 2021/0106717 A1
(43) Pub. Date: Apr. 15, 2021

(54) BONDED POWDERS FOR THE TREATMENT OF BODILY LESIONS

(71) Applicant: Cook Medical Technologies LLC, Bloomington, IN (US)

(72) Inventors: Shaun D. Gittard, Winston-Salem, NC (US); John C. Sigmon, JR., Winston-Salem, NC (US); Christopher A. Carruthers, Winston-Salem, NC (US)

(73) Assignee: Cook Medical Technologies LLC, Bloomington, IN (US)

(21) Appl. No.: 16/598,189

(22) Filed: Oct. 10, 2019

(51) Int. Cl. A61L 24/02; A61M 5/142; A61K 9/14; A61K 31/78; A61K 33/06; A61K 33/10; A61L 24/06

(52) U.S. Cl. CPC A61L 24/02; A61M 5/142; A61K 9/14; A61L 2400/04; A61K 33/06; A61K 33/10; A61L 24/06; A61K 31/78

(57) ABSTRACT

The present embodiments provide systems and medical formulations suitable for delivering therapeutic powders to a target site. In one embodiment, the system comprises a delivery device, a first powder being formed of particles of a first material, and a second powder being formed of particles of a second material, wherein the second material is different than the first material. At least some of the particles of the first powder and the second powder are bound together to form bonded particles. The bonded particles are simultaneously delivered to the target site by the delivery device. In one embodiment, a medical formulation may comprise carbomer present in a range between about 60-80% by weight of the formulation, bentonite present in a range of between about 5-15% by weight of the formulation, and calcium carbonate present in a range of between about 10-30% by weight of the formulation.

### BACKGROUND

The present embodiments relate generally to medical products, and more particularly, to formulations, systems and methods for treating a bodily lesion.

There are several instances in which it may become desirable to introduce therapeutic agents into the human or animal body. For example, therapeutic drugs or bioactive materials may be introduced to achieve a biological effect. The biological effect may include an array of targeted results, such as inducing hemostasis, sealing perforations, reducing restenosis likelihood, or treating cancerous tumors or other diseases.

Localized delivery of therapeutic agents has been performed using catheters and similar introducer devices. By way of example, a catheter may be advanced towards a target site within the patient, then the therapeutic agent may be injected through a lumen of the catheter to the target site. Typically, a syringe or similar device may be used to inject the therapeutic agent into the lumen of the catheter. However, such a delivery technique may result in a relatively weak stream of the injected therapeutic agent.

Moreover, it may be difficult or impossible to deliver therapeutic agents in a targeted manner in certain forms, such as a powder form, to a desired site. For example, if a therapeutic powder is held within a syringe or other container, it may not be easily delivered through a catheter to a target site in a localized manner that may also reduce potentially harmful side effects.

With regard to the gastrointestinal tract in particular, there are several conditions that may cause lesions requiring localized treatment. For example, gastrointestinal inflammation, gastrointestinal cancer, gastrointestinal infection, gastrointestinal motility dysfunction, or lesions, wounds or contusions of tissue of a portion of the gastrointestinal tract that can cause gastrointestinal lesions. In addition, there are a wide variety of medical procedures that require removal of the mucosal or submucosal layers of the gastrointestinal tract wall and can also cause injury or lesions in the gastrointestinal tract. These procedures include endoscopic mucosal resection (EMR), endoscopic submucosal dissection, polypectomy, per-oral endoscopic myotomy, biopsy, and ablation (thermal, chemical, radiofrequency, and cryogenic). As with the disorders of the gastrointestinal tract, similar adverse events can occur after removal of the mucosal or submucosal layers, such as bleeding.

The use of mucoadhesives and bioadhesives has been known to open epithelial tight junctions, prevent intestinal ulceration, retain drugs in open wounds, increase ocular-surface residence, and have vaccine adjuvant activity.

Although the use of mucoadhesive agents and hemostatic agents are known to an extent in an isolated context, the delivery of multiple agents together, and particularly in a powder form to a gastrointestinal tract, is a new and challenging area.

### SUMMARY

The present embodiments provide systems and medical formulations suitable for delivering therapeutic powders to a target site. In one embodiment, the system comprises a delivery device, a first powder being formed of particles of a first material, and a second powder being formed of particles of a second material, wherein the second material is different than the first material. At least some of the particles of the first powder and the second powder are bound together to form bonded particles prior to being placed into the delivery device. The bonded particles of the first and second powders are simultaneously delivered to the target site by the delivery device.

In one embodiment, the first powder comprises a mucoadhesive agent and the second powder comprises a hemostatic agent. In one example, the mucoadhesive agent may comprise carbomer, and the hemostatic agent may comprise bentonite. The particles of the first powder and the second powder may be bound together by one of hydrogen bonding, van der Waals bonding, metallic bonding, ionic bonding, covalent bonding, chain entanglement, coating, impaction or embedding. In one embodiment, at least 10% of the particles of the first powder and the second powder are bound together.

In one example, the bonded particles of the first and second powders have a minimum width of at least 40 microns, and have a maximum width of less than 200 microns. A difference between a density of the particles of the first powder and a density of the particles of the second powder may be at least two times. The moisture content of the bonded particles may be between 1% and 50%. In one embodiment, a mass of the bonded particles is in a range of between about 0.0015 mg to about 0.15 mg per particle.

In one example, the delivery device comprises a container for holding the bonded particles, a pressure source having pressurized fluid, the pressure source in selective fluid communication with at least a portion of the container, and a catheter in fluid communication with the container and having a lumen sized for delivery of the bonded particles to a target site. A ratio of an inner diameter of the catheter to the diameter of bonded particles may be at least 4:1.

In one embodiment, the medical formulation comprises a first powder being formed of particles of a first material, wherein the first powder comprises a mucoadhesive agent, and a second powder being formed of particles of a second material, wherein the second powder comprises a hemostatic agent. Particles of the first powder and the second powder are bound together to form bonded particles. The mucoadhesive agent may be present in a greater quantity in the bonded particles than the hemostatic agent. In some embodiments, the mucoadhesive agent may be present in a quantity of at least 50% by weight of the formulation. In one example, the mucoadhesive agent comprises carbomer and the hemostatic agent comprises bentonite.

The medical formulation may further comprise calcium carbonate. In such example, carbomer may be present in a range between about 60-80% by weight of the formulation, bentonite may be present in a range between about 5-15% by weight of the formulation, and calcium carbonate may be present in a range between about 10-30% by weight of the formulation.

An exemplary method for delivering therapeutic powders to a target site comprises providing a first powder being formed of particles of a first material, and a second powder being formed of particles of a second material, wherein the second material is different than the first material. The method includes binding the particles of the first powder and the second powder together to form bonded particles prior to being placed into a delivery device, and then simultaneously delivering the bonded particles of the first and second powders to the target site by the delivery device.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIGS. 1A-1B are views under a scanning electron microscope illustrating particles of two powders that are unbonded and bonded, respectively.

FIG. 2 is a perspective view showing an example of bonded powders suitable for spraying through a catheter.

FIG. 3 is a perspective view showing an example of bonded powders becoming an aerosol during testing exercises.

FIG. 4 is a perspective view showing an example of bonded powders clogging a catheter during testing exercises.

FIGS. 5-6 are perspective views showing examples of a mixed powder without being bound into a compound forming a precipitate, and the same mixed powder being bound and forming a gel, respectively.

FIG. 7 is a side-sectional view of an exemplary delivery system suitable for use with the bonded powders of the present embodiments.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patient's anatomy during a medical procedure.

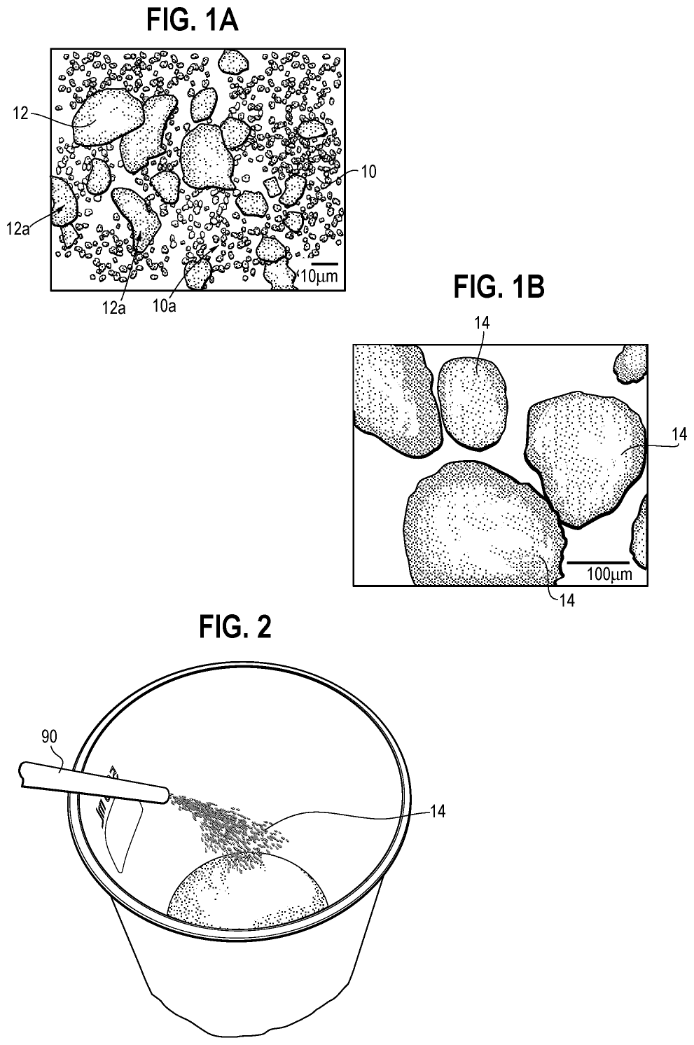

Referring to FIGS. 1A-1B, in a first embodiment, a first powder 10 is formed of particles 10a of a first material, and a second powder 12 is formed of particles 12a of a second material. In FIG. 1A, the particles 10a of the first powder 10 are shown in an unbonded state relative to the particles 12a of the second powder 12. However, in FIG. 1B, the particles 10a of the first powder 10 are shown in a bonded state relative to the particles 12a of the second powder 12, thereby forming bonded particles 14, according to techniques described further below.

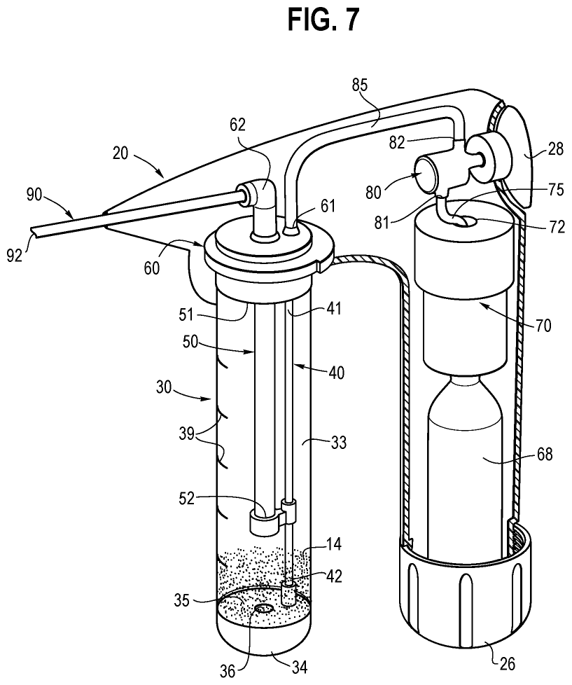

The particles 10a of the first powder 10 and the particles 12a of the second powder 12 are bound together to form the bonded particles 14 prior to being placed into a delivery device, such as the exemplary delivery device 20 shown in FIG. 7 below. The bonded particles 14 comprising the first and second powders 10 and 12 then are simultaneously delivered to a target site by the delivery device 20, as explained further below.

In accordance with one aspect, the particles 12a of the second powder 12 comprise a different composition than the particles 10a of the first powder 10. The powder materials can be any one of the following: mucoadhesive agent, hemostatic agent, adhesive agent, pharmaceutical agent, biologic, polymer, mineral, ceramic, metal, composite, colorant, acid, base, buffer, crosslinker, catalyst, dye, radiopaque agent, nanoparticle, or inert filer. Exemplary biologics include proteins, genes, amino acids, ligands, hormones, and lipids. In one embodiment, for example, where a gastrointestinal lesion is being treated, the first powder 10 may comprise a mucoadhesive agent and the second powder 12 may comprise a hemostatic agent.

As used herein, the term “mucoadhesive agent” refers to an agent that adheres to a mucous membrane, which may line the wall of a body vessel or body cavity, e.g., a gastrointestinal surface, such as either or both of a gastrointestinal epithelia or mucosa (including submucosa) and preferably at or about a site of a lesion. The mucous membrane may include a moist mucous layer to which the mucoadhesive agent may adhere. A mucoadhesive agent may adhere to a mucous membrane by physical and/or chemical forces including, for example, ionic bonding, covalent bonding, hydrogen bonding, Van der Waals bonding, or hydrophobic bonding (i.e., hydrophobic interaction).

One example of a mucoadhesive agent suitable for use herein includes a macromolecule (e.g., a polymer) including repeating monomer units. Other examples of mucoadhesive agents for use in the present embodiments include, for example, a hydrophilic polymer, a hydrogel, a co-polymer, or a thiolated polymer. The hydrogen bond forming functional groups may include carboxyl groups, hydroxyl groups, carbonyl groups, sulphate groups, amide groups, or any other functional groups capable of forming hydrogen bonds. Examples of mucoadhesive agents or components thereof may include, for example, carbomers (e.g., polyacrylic acids), polycyclic aromatic hydrocarbons (e.g., retene), carboxylic acids, polyvinylpyroolidones, polyvinylalchohols, polycarbophils, chitosan materials (i.e., poliglusam, deacetylchitin, or poly-(D)glucosamine), sodium alginates, cellulose derivatives (e.g., methylcellulose, methylethylcellulose, sodium carboxymethylcellulose, hydroxypropylcellulose, hydroxypropylmethylcellulose, or hydroxyethylcellulose), ethers (e.g., polyethylene glycol), lectins (e.g., Erythrina c. lectin, Concanavalin a. lectin, Ulex europaeus lectin, and C-Type lectin), thiamines (e.g., thiamine end capped polymer chains); pathogenic bacteria (e.g., bacterial fimbrins), thiols (e.g., chitosan-cysteine, chitosan-thiolbutylamidine, chitosan-thioglycolic acid, polyacrylic acid-cysteine, polyacrylic acid-cysteamine, carboxymethylellulose-cystein, or alginate-cysteine), amino acid sequences, ion-exchange resins (e.g., cholestyramine), or any biomolecules including an amino acid sequence (e.g., peptides). Additional examples of mucoadhesive agents or components thereof may include mucin, guar gum, karya gum, xantham gum, locust bean gum, acacia gum, gellan gum, tragacanth, soluble starch, gelatin, or pectin. In some examples, mucoadhesive agents may include any biomolecules having an affinity for mucosal tissue such as, for example, proteins (e.g., fimbrial proteins or affinity ligands). Other types of tissue adhesives include cyanoacrylate glues and sealants, glutaraldehyde, L-DOPA, or any other known polymer or biologic adhesives.

As used herein, the term “hemostatic agent” refers to an agent that has properties amenable to facilitating hemostatis of tissue. By way of non-limiting example, a hemostatic agent may comprise alginate, smectite clays, chitin, chitosan, collagen, fibrin, clotting factors, kaolinite clays, oxidized cellulose, plant-based polysaccharides, platelets, smectite clays, and zeolites.

In accordance with one aspect of the present embodiments, a mucoadhesive agent and a hemostatic agent are bound together and delivered simultaneously to a target site in a manner that achieves advantages of both the mucoadhesive agent and the hemostatic agent, e.g., adhering to a mucous membrane and facilitating hemostasis of tissue. Although such agents are known individually in an entirely isolated context, by bonding such particles together prior to delivery, improved treatment of lesions may be achieved via the bonded composition.

In one embodiment, where the formulation of the first powder 10 comprises a mucoadhesive agent and the second powder 12 comprises a hemostatic agent, the formulation may be delivered to a target site at a short distance, e.g., via a nozzle disposed adjacent to tissue, or via an applicator in direct contact with exposed tissue, e.g., during an open surgical procedure or bleeding of the arms, legs, or other external extremities.

However, in order to expand the application of the formulation comprising particles 10a of the mucoadhesive agent and particles 12a of the hemostatic agent, it is desirable to be able to deliver the formulation a relatively significant distance, such as the length of a delivery catheter being inserted through a conventional endoscope. An exemplary delivery catheter 90 is shown as part of the delivery device of FIG. 7 and described further below. If the formulation is capable of traversing such relatively long distance, then it may be suitable for treatment of gastrointestinal lesions, e.g., via delivery through a lumen 92 of the catheter 90 that in turn is extended through a lumen of an endoscope.

As will be explained further below, after extensive experimental testing, the inventors have concluded that having particles of the mucoadhesive agent and the hemostatic agent being bound together as bonded powders 14, as opposed to being delivered separately unbonded, provides particular advantages. For example, if one particle is lighter, then when delivered separately (unbonded) there exists a higher likelihood of preferential delivery of the heavier constituent to the target site while the other remained aerosolized. By bonding at least some of the particles of the different materials together, as explained further below, the risk of adverse events such as aerosolization of one particle may be reduced, increasing the likelihood that both particles reach the target site if other properties below are appropriate.

The bonded powders 14 preferably have a specific range of properties that make them suitable for delivery through the catheter 90, particularly when the catheter 90 is sized for delivery through a lumen of an endoscope. As one example, the mass of an individual particle of the bonded powders 14 should be within a specific range. If particles of the bonded powders 14 are too heavy, it will require too much pressure to travel the length of the catheter 90 and can result in clogging of the catheter 90. On the other hand, if particles of the bonded powders 14 are too light, it will aerosolize within the patient's body, e.g., in the gastrointestinal space, instead of being propelled to a target site.

In addition to mass of an individual particle of the bonded powders 14, the size of the collective bound particles is important for ensuring proper delivery through the catheter 90. If the bound particles are too large in size, then they will be prone to clogging within the delivery catheter 90. If the bound particles are too small, they may have a higher likelihood of being aerosolized instead of being propelled to the target site.

In one embodiment, it has been found beneficial to have bound particles of the bonded powder 14 comprise a diameter in the range of about 1 micron to about 925 microns, and preferably in the range of about 40 microns to about 200 microns. Further, it has been found highly beneficial to have the bound particles of the bonded powder 14 comprise a mass in the range of about 0.0001 mg to about 0.5 mg per bonded particle, and preferably in the range of about 0.0015 mg to about 0.15 mg per bonded particle. It has been determined through multiple testing exercises that such ranges have criticality in terms of significantly reducing the likelihood of clogging of the catheter 90 during delivery, and also significantly reducing the likelihood of having the bonded particles aerosolize during delivery, and therefore be properly delivered to a target site in the correct dose.

Particles of the bonded powder 14 may be ground, compacted and/or sieved to produce the desired particle size and mass. As used herein, particle mass is dependent on the density of the materials and the volume of the particle. Further, regarding size, an assumption can be made that the particles are spheres, in which case the diameter ranges noted herein apply. However, it will be appreciated that other particle shapes exist, especially for crystalline materials. If the particle is substantially non-spherical, then similar micron ranges listed herein for spherical particles may apply, but instead of referring to diameter the value may refer to average or maximum width of the particle.

With regard to dimensions of the catheter 90, when used in endoscopic applications, it is clinically important to size the catheter 90 to be small enough to fit through a working lumen of the endoscope, yet be large enough to substantially avoid clogging when the bonded powders 14 are advanced through the catheter. In one embodiment, it has been found beneficial to have a ratio of catheter inner diameter to bound particle diameter to be at least 4:1, and more preferably at least 7.5:1. The inventors have tested various embodiments, and determined that there is criticality in providing the ratio above 4:1, with any suitable size catheter that can be advanced through a lumen of an endoscope.

It should be noted that endoscopes are generally available with accessory channels up to 4.2 mm. Since a catheter inserted through this channel has a wall thickness of generally greater than 0.25 mm, the maximum projected inner diameter of the catheter for endoscopic delivery would be 3.7 mm. Based on a 4:1 ratio of catheter inner diameter to particle diameter, then the maximum acceptable particle diameter would be approximately 925 microns. Further, it is noted that spherical particles may be less susceptible to clogging than cuboid or flat particles. Accordingly, a ratio of closer to 4:1 may be acceptable for spherical particles, whereas a higher ratio (e.g., 7.5:1 or greater) may be preferable for other particle shapes.

With regard to pressure, a pressure source 68 of the delivery device 20, as depicted in FIG. 7 below, may comprise a pressurized fluid cartridge of a selected gas or liquid, such as carbon dioxide, nitrogen, or any other suitable gas or liquid that may be compatible with the human body. The pressurized fluid cartridge may contain the gas or liquid at a relatively high, first predetermined pressure, for example, around 1,800 psi inside of the cartridge. The pressure source may be in a solid (dry ice), liquid or gas state. As further noted above, the fluid may flow from the pressure source 68 through a pressure regulator, such as regulator valve 70 having a pressure outlet 72 leading to outlet tubing 75, which may reduce the pressure to a lower, second predetermined pressure (referred to here as a “delivery system pressure”). In one embodiment, it has been found beneficial to have a delivery system pressure in the range of about 0.01 psi to about 100 psi, and preferably in the range of about 0.5 psi to about 75 psi. It has been determined through multiple testing exercises that such ranges have criticality in terms of providing appropriate force to propel the bonded powder 14 through the catheter 90, while significantly reducing the likelihood of clogging of the catheter 90 during delivery, and therefore properly deliver the bonded powder 14 to a target site in the correct dose.

In FIG. 7, further details of one embodiment of the delivery device 20 suitable for delivering bonded powders 14 is shown. In this embodiment, the device 20 comprises a container 30 having a reservoir 33 that is configured to hold the bonded powder 14, and further comprises the pressure source 68 that is configured to be placed in selective fluid communication with at least a portion of the container 30, to deliver the bonded powder 14 through a catheter 90 to a target site within the patient. The container 30 may comprise measurement indicia 39, an inlet tube 40, an outlet tube 50, and a cap 60. The inlet tube 40 has first and second ends 41 and 42 with a lumen extending therebetween, while the outlet tube 50 has first and second ends 51 and 52 with a lumen extending therebetween. The first end 41 of the inlet tube 40 is placed in fluid communication with an inlet port 61 formed in the cap 60, while the first end 51 of the outlet tube 50 is placed in fluid communication with an outlet port 62 formed in the cap 60, as shown in FIG. 7. Fluid passed through the inlet port 61 of the cap 60 is directed through the inlet tube 40, through an opening 36 in a platform 35, and the fluid and the bonded powder 14 within the reservoir 33 may be directed through the outlet tube 50, through the outlet port 62, through the catheter 90, and towards a target site. Actuators 26 and 28 may be engaged by a user and selectively operated to perform the functions related to release of the pressurized fluid from the cartridge 68. An actuation valve 80 comprises an inlet port 81 and an outlet port 82 to selectively permit fluid to pass from the inlet port 81 to the outlet port 82, then into tubing 85, and then into the inlet port 61 of the cap 60. Further information regarding the delivery device 20, including the design shown in FIG. 7 and multiple alternative embodiments also suitable for delivering the bonded powders 14 is described in U.S. Pat. No. 8,118,777, which is hereby incorporated by reference in its entirety.

In view of Newton's Second Law (force equals mass times acceleration), acceleration of a particle of the therapeutic agent is dependent upon the particle mass and force applied to the particle. Therefore, a minimum force is necessary to overcome the force of gravity on the particles and to accelerate them to the desired velocity at the time at which they exit the distal end of the catheter 90. It is noted that increases in pressure of the pressure source 68 will deliver the bonded powder 14 more quickly, however, too high of a pressure can cause too high of a particle velocity and subsequently aerosolization.

There is a relationship between particle size, particle mass, and delivery velocity, which can be described by the drag equation: F_{D}=(½)(ρ)(ν²)(C_{D})(A); and the gravitational force equation: F_{G}=(m)(g). In these equations, ρ is the density of air (1.184 kg/m³), ν is the velocity of the particles of the bonded powder 14, C_{D} is the drag coefficient (0.47 if the particles of the bonded powder 14 are assumed to be spherical), A is the cross-sectional area of a particle of the bonded powder 14, m is the mass of a particle of the bonded powder 14, and g is the acceleration due to gravity (9.81 m/s²).

Aerosolization occurs when the drag force exceeds the gravitational force on the particles of the bonded powder 14. Therefore, if the powder delivery velocity is too high relative to the mass of the particles, aerosolization can occur. The shape of the particles and size of the particles also should be factored into account, with more cubic shaped particles and larger particles requiring a lower delivery velocity so they do not aerosolize. In essence, for a given delivery system, there is a minimum particle mass at which aerosolization will occur.

In a preferred embodiment, the system of the present embodiments has a gravitational force F_{G} to drag force F_{D} ratio of preferably greater than 1:1. However, as the velocity of the particles of the bonded powder 14 rapidly decreases with drag force, systems with gravitational force F_{G} to drag force F_{D} ratios as small as 0.001:1 will clear within less than a minute.

Referring to FIG. 2, an example of bonded powders 14 suitable for spraying through a catheter 90 is shown. The bonded powders comprise the formulations disclosed herein, and the desired property ranges noted above for mass of an individual bonded particle, diameter of an individual bonded particle, pressure from the source 68, and the ratio of the size of the bonded particle relative to the inner diameter of the catheter 90.

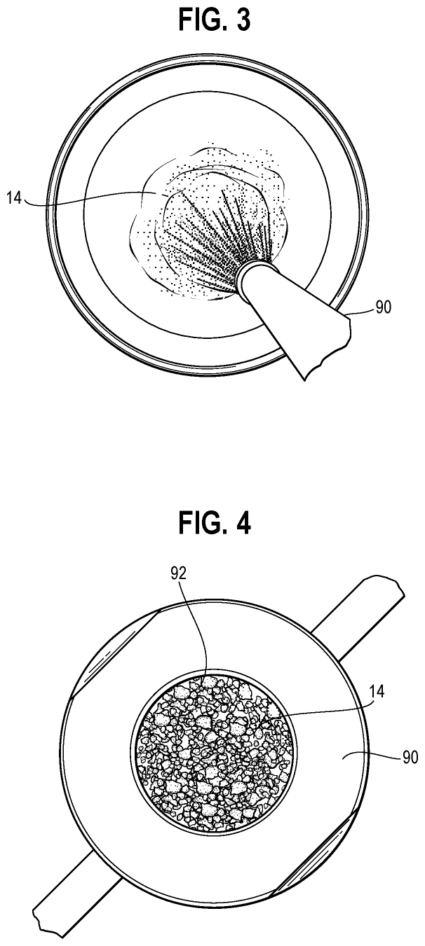

Referring to FIG. 3, an example of bonded powders 14 becoming an aerosol during extensive testing exercises is shown. In this example, the bonded powders 14 travel a portion of a length of the delivery catheter 90, but have become an aerosol prior to intended delivery to a target site, e.g., due to the particles of the bonded powders 14 being too light and/or too small.

Referring to FIG. 4, on the other hand, an example of a bonded powder 14 clogging the catheter 90 during testing exercises is shown. In this example, the bonded powder 14 could not successfully travel the length of the catheter 90 without clogging within its lumen 92, e.g., due to the particles of the bonded powders 14 being too large in diameter and/or too heavy.

As noted above, in accordance with one aspect of the present embodiments, a mucoadhesive agent and a hemostatic agent are bound together and delivered simultaneously to a target site in a manner that achieves advantages of both the mucoadhesive agent and the hemostatic agent, e.g., adhering to a mucous membrane and facilitating hemostasis of tissue. However, given the numerous options of agents that could have been selected for either the mucoadhesive agent or the hemostatic agent (see, e.g., listing of exemplary agents above), it was difficult to select a particular combination of a mucoadhesive agent and a hemostatic agent that could be bound together in a manner suitable for simultaneous delivery to a target site, without becoming an aerosol or clogging a catheter as explained above.

The inventors tested numerous combinations of mucoadhesive agents and hemostatic agents, and concluded that in one example of a highly beneficial bonded powder 14, the mucoadhesive agent comprises carbomer and the hemostatic agent comprises bentonite. Referring back to FIGS. 1A-1B, in this example, the particles 10a of the first powder 10 are carbomer, and the particles 12a of the second powder 12 are bentonite.

In the embodiment where the particles 10a are carbomer and the particles 12a are bentonite, the carbomer and bentonite particles may be bound together by one of hydrogen bonding, van der Waals bonding, metallic bonding, ionic bonding, covalent bonding, chain entanglement, coating, impaction or embedding. In one embodiment, at least 10% of the particles of the first powder 12 and the second powder 14 are bound together.

In an initial testing iteration, the first powder 10 comprising carbomer and the second powder 12 comprising bentonite were unable to be delivered to a target site as a spray driven by carbon dioxide due to aerosolization. It is noted that bentonite has a higher density that the carbomer used in this experiment, yielding concern of preferential delivery of the heavier constituent to the target site while the other remained aerosolized.

In a subsequent testing iteration, the first powder 10 comprising carbomer and the second powder 12 comprising bentonite were bound together and filtered to a specific particle range size. The particle size resulted in a compound that could be effectively delivered to a target sire without significant loss due to aerosolization, clogging the catheter, or being too large and heavy to effectively spray.

In a preclinical survival study in a porcine animal model, it was found that the compound comprising carbomer and bentonite may not be durable due to the presence of salts. In a series of further experimental testing, calcium carbonate was subsequently included in the formulation as additional base to increase the amount of gelation by locally neutralizing the acidic environment resulting in a swelled and more viscous gel that was less susceptible to breakdown from the presence of salinity. However, divalent inorganic bases such as calcium carbonate ionically crosslink the carbomer causing precipitation.

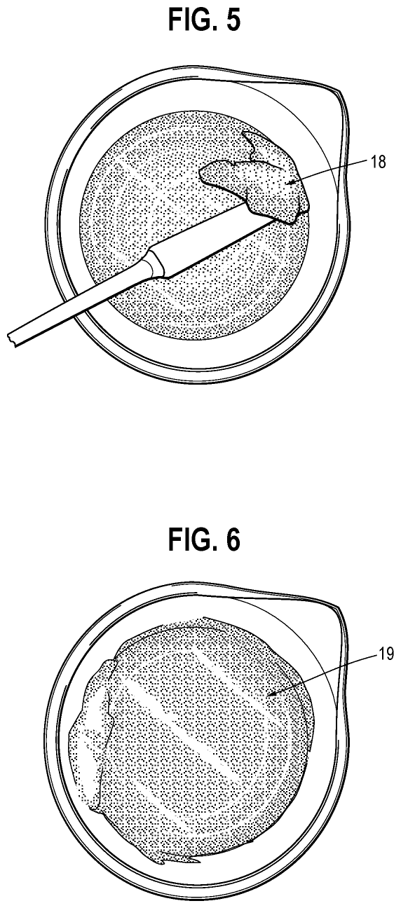

Bonding the particles together was discovered by the inventors to immobilize the divalent cations of the buffer within the resultant compound, preventing the formation of a precipitate in solution. FIG. 5 shows an example of a mixed powder without being bound into a compound forming a precipitate 18, while FIG. 6 shows the same mixed powder being bound and forming a gel 19.

Calcium carbonate was selected as a desirable base after multiple different bases were compared in a benchtop drip test. The compound permutated with the addition of different bases were placed on small intestinal submucosa and subsequently exposed to a dripped saline solution over time via an in-line peristalic pump. The subsequent survival study found that the modified compound was durable.

In one presently preferred embodiment, the medical formulation comprises carbomer present in a range between about 60-80% by weight of the formulation, bentonite present in a range of between about 5-15% by weight of the formulation, and calcium carbonate present in a range of between about 10-30% by weight of the formulation.

While the above ranges are not strictly limiting, there is a preference for the mucoadhesive agent to be present in a greater quantity in the bonded particles than the hemostatic agent. Thus, the mucoadhesive agent is desirably present in a quantity of at least 50% in the bonded particles. In such embodiment, the selection to have the mucoadhesive agent present in a quantity of at least 50% in the bonded particles is based on clinical testing, particularly from a standpoint of adhesion performance.

In embodiments where the powders may be hygroscopic, the moisture content should be controlled prior to application to the site of interest. In such embodiments, the moisture content of the bonded composition may be between 1% and 50%. It is noted that too much moisture may reduce adhesion performance, and strong adhesion may be desirable when treating lesions, particularly in the gastrointestinal tract.

Advantageously, the present formulation provides excellent performance for all areas of the gastrointestinal tract. In particular, the gelation with calcium carbonate present in a range of between about 10-30% by weight of the formulation reduces breakdown of the formulation due to gastrointestinal properties. In short, the same formulation of bonded powders 14 can be used in different gastrointestinal areas without the need to switch out the specific powders 10 and 12 for different applications.

As a further advantage of the present embodiments, there is provided the ability to simultaneously deliver two materials of differing densities to a target site using spraying techniques, e.g., using the delivery device 20. The difference between a density of the particles 10a of the first powder 10 and a density of the particles 12b of the second powder 10 may be at least two times. Such density distinction applies where the first powder 10 comprises carbomer and the second powder 12 comprises bentonite. Given the varying densities, it was challenging to deliver the bonded powder 14 by spraying without aerosolizing or clogging a catheter. However, after the extensive trials disclosed herein, a suitable formulation (particularly one comprising carbomer, bentonite, and calcium carbonate, and desirably in the proportions noted above), combined with the technique for binding the formulation, coupled with extensive testing regarding an appropriate mass, diameter, delivery pressure and catheter to bonded particle ratio as explained above, have overcome the aerosolization and clogging challenges associates with simultaneously delivering two materials of differing densities to a target site using spraying techniques. Moreover, the suitable formulation, in which the mucoadhesive agent is present in a quantity of at least 50% by weight of the formulation, provides excellent adhesion performance, particular to gastrointestinal tissue.

With regard to the properties described above, it should be noted that while they have been generally described with respect to the system of FIG. 7, i.e., for use with a catheter 90 suitable for endoscopic delivery, it will be appreciated that these combinations of particle properties, catheter to particle ratios, delivery system pressure, and other properties may be used in conjunction with different agent delivery systems apart from the device depicted in FIG. 7. For example, the above-referenced properties may be beneficial for any delivery of a bonded powder through a catheter, even when the catheter is not delivered through an endoscope.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

1. A system suitable for delivering therapeutic powders to a target site, the system comprising:
a delivery device;
a first powder being formed of particles of a first material; and
a second powder being formed of particles of a second material, wherein the second material is different than the first material,
wherein at least some of the particles of the first powder and the second powder are bound together to form bonded particles prior to being placed into the delivery device,
wherein the bonded particles of the first and second powders are simultaneously delivered to the target site by the delivery device,
wherein the first powder comprises a mucoadhesive agent and the second powder comprises a hemostatic agent, and
wherein the mucoadhesive agent is present in a greater quantity by weight of the formulation than the hemostatic agent.

2. The system of claim 1, wherein the mucoadhesive agent is present in a quantity of at least 50% by weight of the formulation.

3. The system of claim 1, wherein the mucoadhesive agent comprises carbomer.

4. The system of claim 1, wherein the hemostatic agent comprises bentonite.

5. The system of claim 1, wherein the particles of the first powder and the second powder are bound together by one of hydrogen bonding, van der Waals bonding, metallic bonding, ionic bonding, covalent bonding, chain entanglement, coating, impaction or embedding.

6. The system of claim 1, wherein at least 10% of the particles of the first powder and the second powder are bound together.

7. The system of claim 1, wherein the bonded particles of the first and second powders have a minimum width of at least 40 microns.

8. The system of claim 1, wherein the bonded particles of the first and second powders have a maximum width of less than 200 microns.

9. The system of claim 1, wherein a difference between a density of the particles of the first powder and a density of the particles of the second powder is at least two times.

10. (canceled)

11. The system of claim 1, wherein a mass of the bonded particles is in a range of between about 0.0015 mg to about 0.15 mg per particle.

12. The system of claim 1, wherein the delivery device comprises:
a container for holding the bonded particles;
a pressure source having pressurized fluid, the pressure source in selective fluid communication with at least a portion of the container; and
a catheter in fluid communication with the container and having a lumen sized for delivery of the bonded particles to a target site.

13. The system of claim 12, wherein a ratio of an inner diameter of the catheter to the diameter of bonded particles is at least 4:1.

14. A medical formulation for protecting or treating a lesion, comprising:
a first powder being formed of particles of a first material, wherein the first powder comprises a mucoadhesive agent; and
a second powder being formed of particles of a second material, wherein the second powder comprises a hemostatic agent,
wherein the particles of the first powder and the second powder are bound together to form bonded particles, and wherein the mucoadhesive agent is present in a greater quantity by weight of the formulation than the hemostatic agent.

15. The medical formulation of claim 14, wherein the mucoadhesive agent is present in a quantity of at least 50% by weight of the formulation.

16. The medical formulation of claim 14, wherein the mucoadhesive agent comprises carbomer and the hemostatic agent comprises bentonite.

17. The medical formulation of claim 16, further comprising calcium carbonate.

18. The medical formulation of claim 17, wherein the carbomer is present in a range between about 60-80% by weight of the formulation, the bentonite is present in a range of between about 5-15% by weight of the formulation, and the calcium carbonate is present in a range of between about 10-30% by weight of the formulation.

19. (canceled)

20. (canceled)

21. A system suitable for delivering therapeutic powders to a target site, the system comprising:
a delivery device;
a first powder being formed of particles of a first material; and
a second powder being formed of particles of a second material, wherein the second material is different than the first material,
wherein at least some of the particles of the first powder and the second powder are bound together to form bonded particles prior to being placed into the delivery device,
wherein the bonded particles of the first and second powders are simultaneously delivered to the target site by the delivery device, and
wherein the bonded particles have predetermined maximum size parameters before being placed into the delivery device.

22. The system of claim 21, wherein the bonded particles are filtered to a specific particle range size before being placed into the delivery device.
